(19) United States
(12) Patent Application Publication
(10) Pub. No.: US 2006/0243378 A1
(43) Pub. Date: Nov. 2, 2006

(54) Absorbent garment and process for making such an absorbent garment

(75) Inventor: Joseph Richard Alberts, Greenville, WI (US)

Correspondence Address: SENNIGER POWERS (KCC), ONE METROPOLITAN SQUARE, 16TH FLOOR, ST LOUIS, MO, 63102, US

(73) Assignee: Kimberly-Clark Worldwide, Inc., Neenah, WI (US)

(21) Appl. No.: 11/119,036

(22) Filed: Apr. 29, 2005

(51) Int. Cl. A61F 13/15

(52) U.S. Cl. 156/256; 604/396; 156/267; 156/204

(57) ABSTRACT

In an absorbent garment and process for making the same, at least one garment shell web is configured in a generally open configuration, an outer cover web is positioned on the garment shell web, an absorbent body web is positioned on the outer cover web and a bodyside liner web is positioned on the absorbent body web. The outer cover web is secured to the inner surface of the at least one garment shell web. The at least one garment shell web is configured in a closed configuration to define a closed configuration of a garment shell of the absorbent garment. In particular, the at least one garment shell web is folded such that the inner surface of the at least one garment shell web defines an interior space of the garment shell and the bodyside liner web, outer cover web and absorbent body web are disposed within the interior space of the garment shell.

### BACKGROUND

The present invention relates generally to absorbent garments, and more particularly to absorbent garments having the appearance of conventional clothing, and even more particularly to a process for making such absorbent garments.

Personal wear garments and other articles find widespread use as personal care products including, without limitation, diapers, children's toilet training pants, adult incontinence garments, sanitary napkins and the like, as well as surgical bandages and sponges. The primary purpose of such garments is to take in and retain body exudates released by a wearer to thereby prevent soiling of the wearer's or caregiver's clothing. Certain absorbent garments are suitably disposable in that they are intended to be discarded after a limited period of use, i.e., the garments are not intended to be laundered or otherwise restored for reuse. Disposable absorbent garments typically comprise an absorbent structure disposed between a liner, which contacts the wearer's skin, and an outer cover, which inhibits liquid body waste absorbed by the absorbent structure from leaking out of the garment. The liner of the absorbent garment is typically liquid permeable to permit liquid body waste to pass therethrough for absorption by the absorbent structure.

In particular absorbent garments, such as those worn about one's waist or lower torso (e.g., diapers, children's training pants, adult incontinence briefs) various attempts have been made to make the garments more visually appealing, such as by applying certain graphics or other features which make the articles appear more like conventional clothing, and more particularly like conventional undergarments. In other absorbent garments an absorbent assembly is covered by a surrounding garment shell that has the appearance of conventional garments or undergarments such as shorts, boxer shorts, briefs, swim trunks, skirt, skort and the like. That is, the absorbent assembly acts similar to conventional absorbent garments to take-in and retain body exudates while the outer garment shell has a more loose fitting appearance, particularly about the legs of the wearer.

One example of a known process for making absorbent garments that include an absorbent assembly disposed within a garment shell involves forming the absorbent assembly and the garment shell separate from each other, inserting the absorbent assembly within the garment shell and then securing the absorbent assembly to the garment shell. One disadvantage of such a process is that two or more independent assembly lines are required to commercially produce the separate absorbent assembly and garment shell and then further assemble them together. This substantially slows the manufacturing process.

Accordingly, there is a need for a more efficient process for making an absorbent garment in which an absorbent assembly is disposed within a garment shell, and more particularly for such a process in which the absorbent assembly is formed substantially concurrently with the garment shell instead of independently thereof.

### SUMMARY

In general, a process according to one embodiment of the present invention for making an absorbent garment for personal wear about a wearer's waist comprises configuring at least one garment shell web in a generally open configuration, with the at least one garment shell having an inner surface and an outer surface. An outer cover web is positioned on the inner surface of the at least one garment shell web while the at least one garment shell web is in the open configuration. An absorbent body web is positioned on the outer cover web, and a bodyside liner web is positioned on the absorbent body web with the bodyside liner web having at least one of a length and a width greater than a corresponding one of a length and width of the absorbent body web such that a portion of the bodyside liner web extends outward of the absorbent body web in generally opposed relationship with the outer cover web. At least a portion of the outer cover web is secured to the inner surface of the at least one garment shell web while the at least one garment shell web is in its open configuration. The garment shell web is configured in a closed configuration thereof to define the closed configuration of the garment shell of the absorbent garment. In particular, the at least one garment shell web is folded about the outer cover web, absorbent body web and bodyside liner web such that the inner surface of the at least one garment shell web defines at least in part the interior space of the at least one garment shell and the outer cover web, absorbent body web and bodyside liner web are disposed within the interior space of the garment shell and together define the absorbent assembly of the absorbent body.

In another embodiment, the process generally comprises configuring at least one garment shell web in a generally open configuration, with the at least one garment shell web having an inner surface and an outer surface. A first outer cover web is positioned on the at least one garment shell web and a second outer cover web is positioned on the at least one garment shell web. The first and second outer cover webs are secured to the at least one garment shell web, and the first outer cover web is secured to the second outer cover web to define at least one seam along which the first and second outer cover webs are secured. The garment shell web is configured in a closed configuration thereof to define the closed configuration of the garment shell of the absorbent garment. This step comprises folding the at least one garment shell web about the first and second outer cover webs such that the inner surface of the at least one garment shell web defines at least in part the interior space of the garment shell. The first and second outer cover webs are disposed within the interior space of the garment shell and at least in part define the absorbent assembly of the absorbent garment.

In yet another embodiment, the process comprises configuring first and second garment shell webs in generally opposed relationship with each other in a generally open configuration of the garment shell webs. The garment shell webs each have an inner surface that at least in part defines the interior space of the garment shell in the closed configuration thereof. A first outer cover web is positioned on the inner surface of the first garment shell web and a second outer cover web, separate from the first outer cover web, is positioned on the inner surface of the second garment shell web. At least one of the first and second outer cover webs is secured to at least one of the first and second garment shell webs. A first absorbent body web is positioned on the first outer cover web and a second absorbent body web is positioned on the second outer cover web. A first bodyside liner web is positioned on the first absorbent body web, and a second bodyside liner web is positioned on the second absorbent body web. The first bodyside liner web is in an opposed relationship with the first outer cover web, and the second bodyside liner web is in an opposed relationship with the second outer cover web. The first and second garment shell webs are configured in a closed configuration thereof to define the closed configuration of the garment shell of the absorbent garment. This step comprises folding the first garment shell web about the first outer cover web, the first absorbent body web and the first bodyside liner web and folding the second garment shell web about the second outer cover web, the second absorbent body web and the second bodyside liner web such that the inner surfaces of the garment shell webs at least in part define the interior space of the garment shell and the first and second outer cover webs, first and second absorbent body webs and first and second bodyside liner webs are disposed within the interior space of the garment shell and together define the absorbent assembly of the absorbent garment.

In accordance with one embodiment of an absorbent garment of the present invention, such an absorbent garment generally comprises a garment shell having a closed configuration in which the garment shell is adapted to encircle a wearer's waist. The garment shell has a waist opening, an interior space and at least one leg opening. An absorbent assembly is disposed at least in part within the interior space of the garment shell for taking in and retaining body exudates released by the wearer. The absorbent assembly has a front waist region, a back waist region and a crotch region extending longitudinally between and interconnecting the front and back waist regions. The absorbent assembly is at least in part secured to the garment shell and comprises an outer cover, a bodyside liner in opposed relationship with the outer cover, and an absorbent disposed between the outer cover and the bodyside liner. The outer cover is constructed of at least two separate webs of material secured together along a seam extending longitudinally generally from the front waist region through the crotch region to the back waist region of the absorbent assembly.

### BRIEF DESCRIPTION OF THE DRAWINGS

FIG. 1 is a front elevation of one embodiment of an absorbent garment of the present invention made in accordance with one embodiment of a process for making such an absorbent garment;

FIG. 2 is a side elevation of the absorbent garment of FIG. 1 with one side of the absorbent garment opened to illustrate interior components of the garment;

FIG. 3 is a vertical section of an absorbent assembly of the absorbent garment of FIG. 2 taken along the line 3-3 of FIG. 2;

FIG. 4 is a perspective of a section of apparatus for making an absorbent garment according to one embodiment of a process for making such an absorbent garment;

FIG. 5 is a perspective of a second section of the apparatus for making an absorbent garment;

FIG. 6 is a perspective of a third section of the apparatus for making an absorbent garment;

FIG. 7 is a perspective of a fourth section thereof;

FIG. 8 is a perspective of a fifth section thereof;

FIG. 9 is a perspective of a sixth section thereof;

FIG. 10 is a perspective of a seventh section thereof; and

FIG. 11 is a perspective of an eighth section thereof.

Corresponding reference characters indicate corresponding parts throughout the drawings.

### DETAILED DESCRIPTION

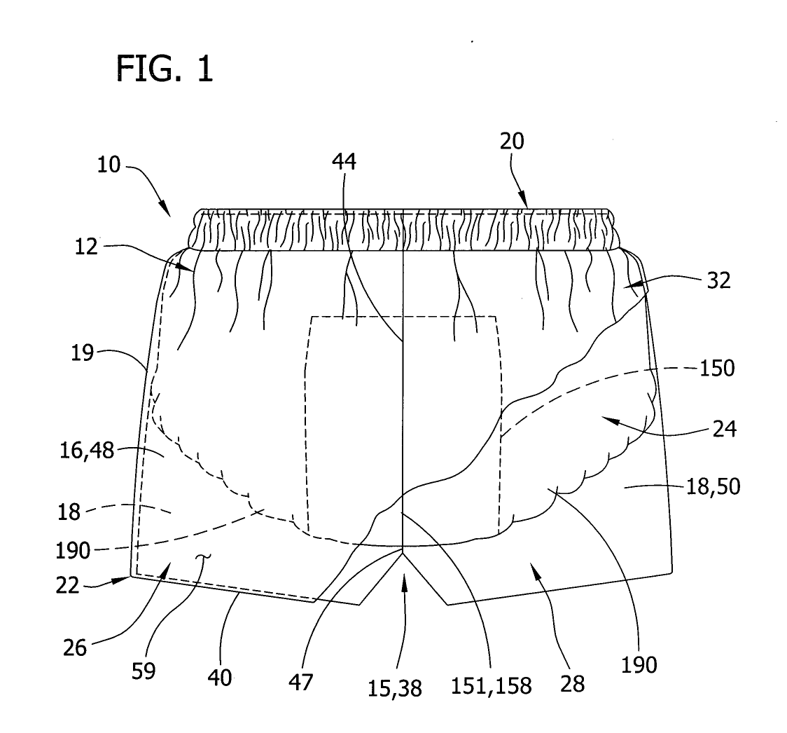

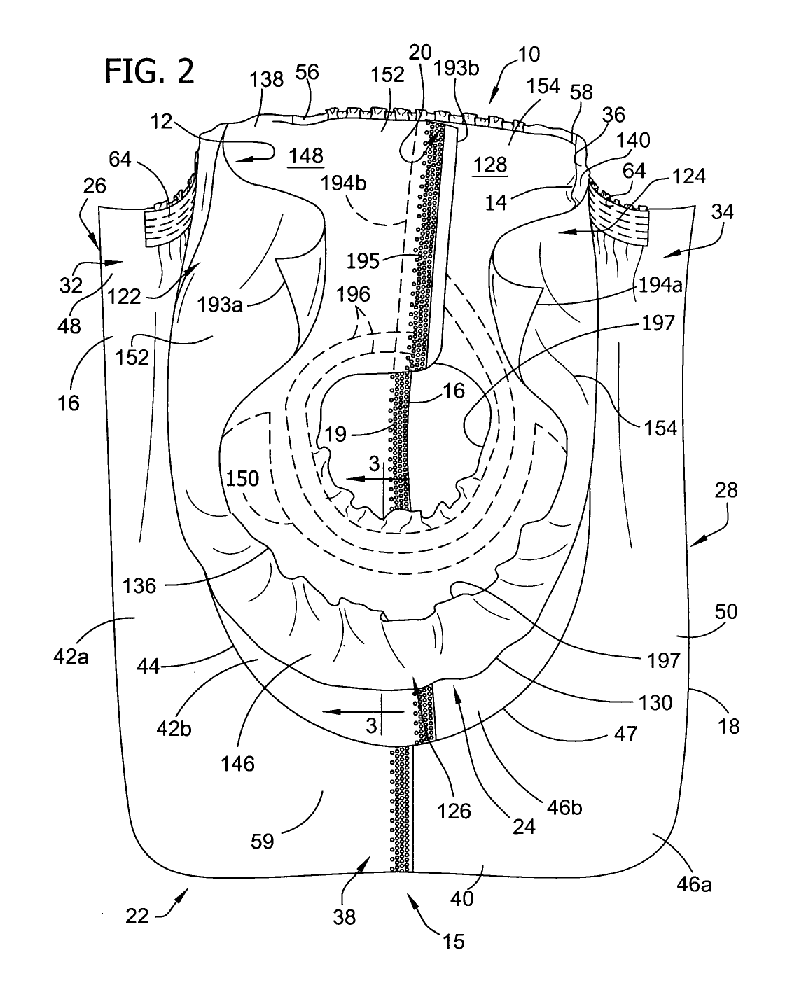

Referring now to the drawings, and in particular to FIGS. 1 and 2, an absorbent garment produced in accordance with one embodiment of a process of the present invention is generally indicated at 10 and is configured for wearing about a wearer's lower torso or waist. The absorbent garment generally has a front waist region, indicated generally at 12, a back waist region, indicated generally at 14 and a crotch region, indicated generally at 15. The front and back waist regions 12, 14 have respective side margins 16, 18 which are attached to each other along side seams 19 of the garment to form a three dimensional configuration of the garment during wear and having a waist opening, generally indicated at 20.

The absorbent garment 10 is suitably configured to resemble conventional clothing such as shorts (e.g., boxer shorts, gym shorts, running shorts, etc.), skirts, skorts (i.e., a combination of a skirt and a pair of shorts), swim trunks and the like, while providing the functions of conventional absorbent articles, such as taking in and retaining body exudates released by the wearer. The absorbent garment 10 comprises a garment shell, generally indicated at 22 and constructed to provide the desired resemblance of the garment to conventional clothing, and an absorbent assembly, generally indicated at 24, disposed within and secured at least in part to the garment shell and constructed to take in and retain body exudates released by the wearer.

With particular reference to FIGS. 1 and 2, the garment shell 22 comprises a front panel assembly, which is generally indicated at 26, having laterally opposite side margins 48 and a back panel assembly, which is generally indicated at 28, having laterally opposite side margins 50. In the illustrated embodiment, the side margins 48 of the front panel assembly 26 broadly define the front side margins 16 of the absorbent garment 10 and the side margins 50 of the back panel assembly 28 broadly define the back side margins 18 of the absorbent garment. As will be described in further detail later herein, the side margins 48, 50 of the front and back panel assemblies 26, 28 of the garment shell 22 are overlapped and attached to each other to broadly define the side seams 19 of the absorbent garment 10, and to define the three-dimensional configuration of the garment shell during wear.

In its three-dimensional configuration (broadly, a closed configuration) as shown in FIGS. 1 and 2, the garment shell 22 has a front waist region 32 which at least in part defines the front waist region 12 of the absorbent garment 10, a back waist region 34 (not visible in FIG. 1) which at least in part defines the back waist region 14 of the absorbent garment, and front and back waist ends, designated 56 and 58, respectively, which together generally define a waist opening 36 of the garment shell. In the illustrated embodiment, the garment shell 22 is configured to resemble a pair of shorts having an interior space 59 and a crotch region 38 extending longitudinally between and interconnecting the front waist region 32 and the back waist region 34 of the garment shell. The crotch region 38 of the garment shell 22 at least in part defines the crotch region 15 of the absorbent garment 10, and also in part defines leg openings 40 of the garment shell (broadly referred to herein as outer leg openings of the absorbent garment). However, it is understood that the crotch region 38 of the garment shell 22 may be omitted (so that the crotch region 15 of the absorbent garment 10 is defined solely by the absorbent assembly 24 as described later herein), such as where the garment shell is intended to resemble a skirt (in which case only one leg opening 40 of the garment shell is provided to accommodate both legs of the wearer), without departing from the scope of this invention.

The front panel assembly 26 of the illustrated garment shell 22 comprises a pair of panel members 42a, 42b which are permanently attached to each other, such as by ultrasonic bonding, thermal bonding, adhesive bonding, stitching or other conventional attachment techniques, along a central seam 44 extending longitudinally from the front waist region 32 to the crotch region 38 of the garment shell. The back panel assembly 28 comprises a pair of panel members 46a, 46b configured and permanently attached to each other in a manner similar to the panel members 42 of the front panel assembly 26 along a central seam 47 (FIG. 1) extending longitudinally from the back waist region 34 to the crotch region 38 of the garment shell 22. It is understood, however, that each of the front and back panel assemblies 26, 28 may be constructed of a single panel member (e.g., of unitary construction) without departing from the scope of this invention.

The panel members 42, 46 of the front and back panel assemblies 26, 28 of the garment shell 22 can be constructed of any suitable material webs, and more suitably a material web that provides a generally cloth-like texture. As an example, the panel members 42, 46 may be constructed from natural and/or synthetic sources and may be constructed in any suitable manner including, but not limited to non-wovens such as spunbond, meltblown, spunbond film laminates, bonded carded web, spunlace, hydroentangled, and needlepunched; knit fabrics such as stretch knit, fleece knit, herringbone knit, jersey knit, raschel knit; and woven fabrics such as broadcloth, twill, percale, poplin, muslin, cambric, chino, flannel, silks and woolens. The panel members 42, 46 are suitably liquid permeable, although it is understood that the panel members may be liquid impermeable without departing from the scope of this invention.

With particular reference to FIG. 2, the front and back panel assemblies 26, 28 of the garment shell 22 are secured to each other at the respective side margins 48, 50 of the panel assemblies, such as by ultrasonic bonding, thermal bonding, adhesive bonding, or other conventional attachment techniques. However, it is contemplated that the front and back panel assemblies 26, 28 of the garment shell may be releasably and refastenably secured to each other, such as by suitable hook and loop fastening systems or other suitable refastenable fastening systems. It is also understood that the front and back panel assemblies 26, 28 may be secured to each other at the respective side margins 48, 50 thereof along the entire length of the side margins, or only a segment of the length of the side margins, within the scope of this invention.

To further enhance the appearance of the absorbent garment 10 as well as the fit of the absorbent garment on the wearer's waist, elastic members 64 (e.g., waistband elastics) are operatively joined to the front and back panel assemblies 26, 28 generally at the respective waist ends 56, 58 thereof. The elastic members 64 can be operatively joined to the garment shell 22 while in a stretched condition so that upon retraction the elastic members gather the garment shell at the front and back waist ends 56, 58 to provide a gathered appearance and to further provide an elastic fit of the absorbent garment on the wearer's waist. The elastic members 64 may extend across the full width of the garment shell or extend across only a portion of the garment shell at the waist ends 56, 58 thereof.

With further reference to FIG. 2, the absorbent assembly 24 comprises a front waist region, generally indicated at 122, a back waist region, generally indicated at 124, a crotch region, generally indicated at 126 interconnecting the front and back waist regions, an inner surface 128, configured for contiguous relationship with the wearer, and an outer surface 130 opposite the inner surface. The front waist region 122 comprises the portion of the absorbent assembly which, when the absorbent garment 10 is worn, is positioned on the front of the wearer while the back waist region 124 comprises the portion of the absorbent assembly which is positioned on the back of the wearer. The crotch region 126 of the absorbent assembly 24 comprises the portion of the assembly which is positioned between the legs of the wearer and covers the lower torso of the wearer. The absorbent assembly 24 also has laterally opposite side edges 136 and longitudinally opposite waist ends, respectively designated herein as front waist end 138 and back waist end 140.

In the illustrated embodiment, the absorbent assembly 24 is a brief-style absorbent assembly such as children's training pants, swim pants or adult incontinence products which are configured for wearing about the full waist of the wearer. That is, the absorbent assembly 24 could be worn on the wearer's waist without being secured to the garment shell 22. It is understood, however, that the absorbent assembly may be generally rectangular in shape, such as I-shaped, T-shaped or other suitable shape, that is not intended to extend about the entire waist of the wearer and remain with the scope of this invention.

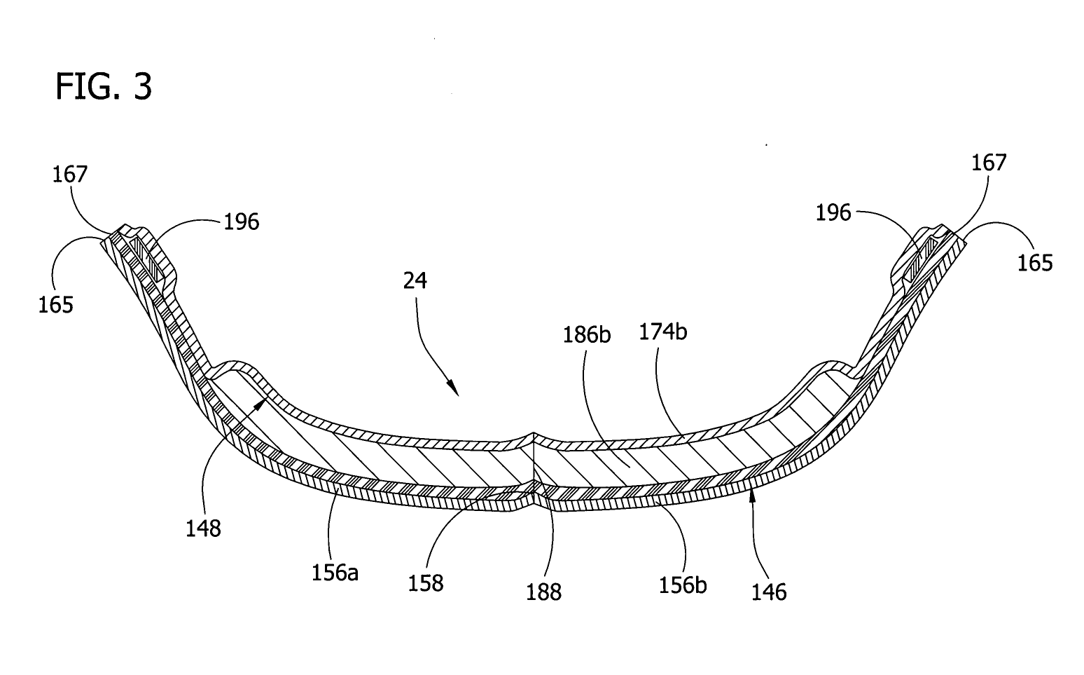

As shown in FIGS. 2 and 3, the absorbent assembly 24 comprises an outer cover, generally indicated at 146, a bodyside liner, generally indicated at 148 in superposed relationship with the outer cover, and an absorbent body, generally indicated at 150, disposed between the outer cover and the bodyside liner. For reasons which will become apparent herein, the absorbent assembly has a seam 151 (FIG. 1) extending longitudinally generally from the front waist region 122 through the crotch region 126 to the back waist region 124 (only the segment of the seam extending down through the front waist region to the crotch region being illustrated in FIG. 1).

The outer cover 146 of the absorbent assembly 24 suitably comprises a material which is substantially liquid impermeable, and may be stretchable or non-stretchable. As used herein, the term “stretchable” refers to a material that may be extensible or elastic. That is, the material may be extended, deformed or the like, without breaking, and may or may not significantly retract after removal of an extending force. As used herein, the term “elastic” refers to that property of a material where upon removal of an elongating force, the material is capable of recovering to substantially its unstretched size and shape or the material exhibits a significant retractive force. The term “extensible” refers to that property of a material where upon removal of an elongating force, the material experiences a substantially permanent deformation or the material does not exhibit a significant retractive force.

More suitably, the outer cover 146 comprises a multi-layered laminate structure in which at least one of the layers is liquid impermeable. For instance, the outer cover 146 can include a liquid permeable outer layer 165 and a liquid impermeable inner layer 167 (FIG. 3) which are suitably joined together by a laminate adhesive, ultrasonic bonds, thermal bonds, or the like. Suitable laminate adhesives, which can be applied continuously or intermittently as beads, a spray, parallel swirls, or the like, can be obtained from Findley Adhesives, Inc., of Wauwatosa, Wis., U.S.A., or from National Starch and Chemical Company, Bridgewater, N.J. U.S.A. The liquid permeable outer layer 165 can be any suitable material and is desirably one that provides a generally cloth-like texture. One example of such a material is a 20 gsm (grams per square meter) spunbond polypropylene nonwoven web. The outer layer 165 may also be made of those materials described later herein from which the liquid permeable bodyside liner 148 is made.

The inner layer 167 of the outer cover 146 can be both liquid and vapor impermeable, or it may be liquid impermeable and vapor permeable. The inner layer 167 can be manufactured from a thin plastic film, although other flexible liquid impermeable materials may also be used. The liquid impermeable inner layer 167 prevents waste material from wetting articles, such as bed sheets and clothing, as well as the wearer and caregiver. A suitable liquid impermeable film for use as a liquid impermeable inner layer 167 of the outer cover 146 is a 0.02 millimeter polyethylene film commercially available from Pliant Corporation of Schaumburg, Ill., U.S.A.

Alternatively, the outer cover 146 may comprise a single layer of liquid impermeable material. As earlier mentioned, the liquid impermeable material can permit vapors to escape from the interior of the disposable absorbent article, while still preventing liquids from passing through the outer cover 146. For example, the outer cover 146 may be constructed of a microporous polymer film or a nonwoven fabric that has been coated or otherwise treated to impart a desired level of liquid impermeability. One such microporous film is a MP-1 film material commercially available from Mitsui Toatsu Chemicals, Inc., Tokyo, Japan, or an XKO-8044 polyolefin film commercially available from 3M Company, Minneapolis, Minn. U.S.A. The single layer outer cover 146 may also be embossed and/or matte finished to provide a more cloth-like appearance.

In the illustrated embodiment, the outer cover 146 of the absorbent assembly 24 is suitably constructed from at least two webs (e.g., left web 156a and right web 156b in the cross-section shown in FIG. 3) that each extend longitudinally of the absorbent assembly. Each of the webs is constructed of the outer cover material or materials described previously. In particular, one outer cover web (e.g., the left web 156a) comprises about one-half of the outer cover 146 and the other outer cover web (e.g., right web 156b) comprises the other half of the outer cover. The webs 156a, 156b are secured together along a seam 158 that extends from the front waist region 122 through the crotch region 126 to the back waist region 124 of the absorbent assembly generally at the lateral centerline of the absorbent assembly and at least in part defines the absorbent assembly seam 151. The outer cover webs 156a, 156b may be secured together by ultrasonic bonding, thermal bonding, adhesive bonding, pressure bonding or other suitable securement technique.

The liquid permeable bodyside liner 148 is illustrated as overlying the outer cover 146 and absorbent body 150, and may but need not have the same dimensions as the outer cover 146. The bodyside liner 148 is desirably compliant, soft feeling, and non-irritating to the child's skin. Further, the bodyside liner 148 can be less hydrophilic than the absorbent body 150, to present a relatively dry surface to the wearer and to permit liquid to readily penetrate through the liner. Alternatively, the bodyside liner 148 can be more hydrophilic or can have essentially the same affinity for moisture as the absorbent body 150 to present a relatively wet surface to the wearer to increase the sensation of being wet. This wet sensation can be useful as a training aid. The hydrophilic/hydrophobic properties can be varied across the length, width and depth of the bodyside liner 148 and absorbent body 150 to achieve the desired wetness sensation or leakage performance.

The bodyside liner 148 can be manufactured from a wide selection of web materials, such as synthetic fibers (for example, polyester or polypropylene fibers), natural fibers (for example, wood or cotton fibers), a combination of natural and synthetic fibers, porous foams, reticulated foams, apertured plastic films, or the like. Various woven and nonwoven fabrics can be used for the bodyside liner 148. For example, the bodyside liner 148 can be composed of a meltblown or spunbonded web of polyolefin fibers. The bodyside liner can also be a bonded-carded web composed of natural and/or synthetic fibers. The bodyside liner 148 can be composed of a substantially hydrophobic material, and the hydrophobic material can, optionally, be treated with a surfactant or otherwise processed to impart a desired level of wettability and hydrophilicity.

For example, the material can be surface treated with about 0.45 weight percent of a surfactant mixture comprising Ahcovel N-62 from Hodgson Textile Chemicals of Mount Holly, N.C., U.S.A. and Glucopan 220UP from Henkel Corporation of Ambler, Pa. in an active ratio of 3:1. The surfactant can be applied by any conventional means, such as spraying, printing, brush coating or the like. The surfactant can be applied to the entire bodyside liner 148 or can be selectively applied to particular sections of the bodyside liner, such as the medial section along the longitudinal center line.

One example of a suitable liquid permeable bodyside liner 148 is a nonwoven bicomponent web having a basis weight of about 27 gsm. The nonwoven bicomponent web can be a spunbond bicomponent web, or a bonded carded bicomponent web. Suitable bicomponent fibers include a polyethylene/polypropylene bicomponent fiber available from CHISSO Corporation, Osaka, Japan. In this particular bicomponent fiber, the polypropylene forms the core and the polyethylene forms the sheath of the fiber. Other fiber orientations are possible, such as multi-lobe, side-by-side, end-to-end, or the like.

In the illustrated embodiment, the bodyside liner 148 of the absorbent assembly 24 is suitably constructed from at least two webs (e.g., left web 174a and right web 174b in the cross-section shown in FIG. 3) that each extend longitudinally of the absorbent assembly. Each of the webs is constructed of the bodyside liner material or materials described previously. In particular, one bodyside liner web (e.g., the left web 174a) comprises about one-half of the liner 148 and the other web (e.g., right web 174b) comprises the other half of the liner. The webs 174a, 174b are secured together along a seam 176 that extends from the front waist region 122 through the crotch region 126 to the back waist region 124 of the absorbent assembly 24 generally at the lateral centerline of the absorbent assembly and at least in part defines the absorbent assembly seam 151. For example, the seam 176 is illustrated as lying on the seam 158 of the outer cover 146.

The bodyside liner webs 174a, 174b may be secured together by ultrasonic bonding, thermal bonding, adhesive bonding, pressure bonding or other suitable securement technique.

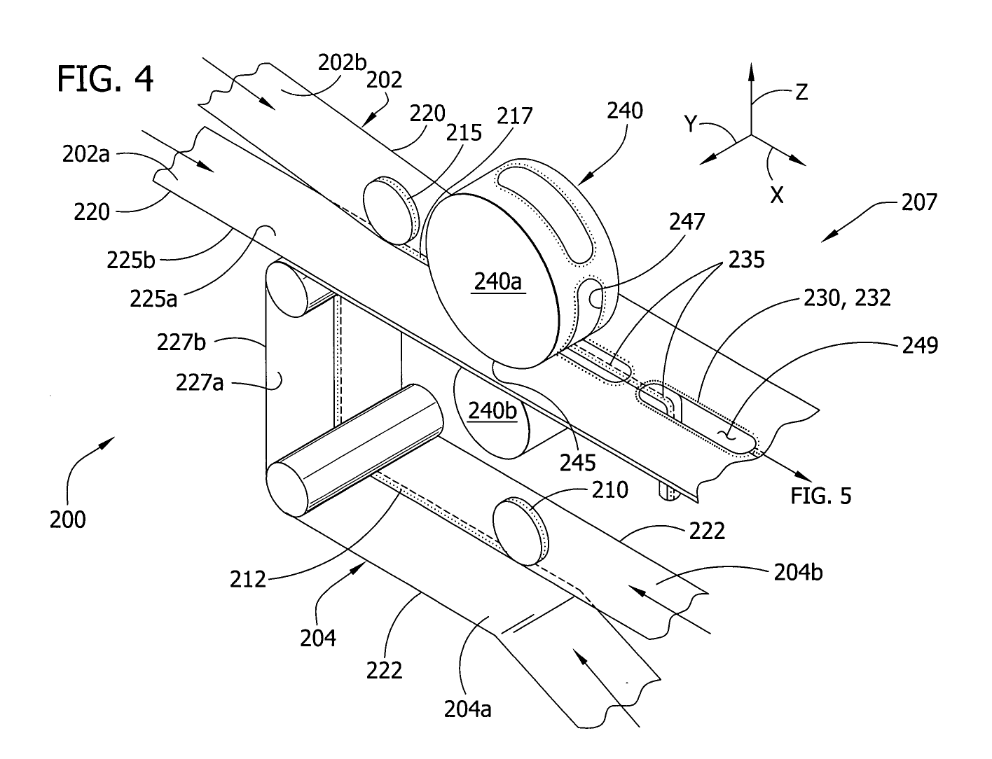

The absorbent body 150 (FIG. 4) is positioned between the outer cover 146 and the bodyside liner 148, which can be joined together by any suitable means such as adhesives, ultrasonic bonds, thermal bonds, or the like. The absorbent body 150 can be any structure which is generally compressible, conformable, non-irritating to the child's skin, and capable of absorbing and retaining liquids and certain body wastes, and may be manufactured in a wide variety of sizes and shapes, and from a wide variety of liquid absorbent materials commonly used in the art. For example, the absorbent body 150 can suitably comprise a matrix of hydrophilic fibers, such as a web of cellulosic fluff, mixed with particles of a high-absorbency material commonly known as superabsorbent material.

In a particular embodiment, the absorbent body 150 comprises a matrix of cellulosic fluff, such as wood pulp fluff, and superabsorbent hydrogel-forming particles. The wood pulp fluff can be exchanged with synthetic, polymeric, meltblown fibers or short cut homofil bicomponent synthetic fibers and natural fibers. The superabsorbent particles can be substantially homogeneously mixed with the hydrophilic fibers or can be nonuniformly mixed. The fluff and superabsorbent particles can also be selectively placed into desired zones of the absorbent body 150 to better contain and absorb body exudates. The concentration of the superabsorbent particles can also vary through the thickness of the absorbent body 150. Alternatively, the absorbent body 150 can comprise a laminate of fibrous webs and superabsorbent material, a foam or other suitable web construction.

Suitable superabsorbent materials can be selected from natural, synthetic, and modified natural polymers and materials. The superabsorbent materials can be inorganic materials, such as silica gels, or organic compounds, such as crosslinked polymers, for example, sodium neutralized polyacrylic acid. Suitable superabsorbent materials are available from various commercial vendors, such as Dow Chemical Company located in Midland, Mich., U.S.A., and Stockhausen GmbH & Co. KG, D-47805 Krefeld, Federal Republic of Germany. Typically, a superabsorbent material is capable of absorbing at least about 10 times its weight in water, and suitably is capable of absorbing more than about 25 times its weight in water.

In one embodiment, the absorbent body 150 comprises a blend of wood pulp fluff and superabsorbent material. One suitable type of pulp is identified with the trade designation CR1654, available from U.S. Alliance, Childersburg, Ala., U.S.A., and is a bleached, highly absorbent sulfate wood pulp containing primarily soft wood fibers and about 16 percent hardwood fibers. In general, the superabsorbent material is present in the absorbent body 150 in an amount of from 0 to about 90 weight percent based on total weight of the absorbent assembly. The absorbent body 150 may or may not be wrapped or encompassed by a suitable tissue wrap that aids in maintaining the integrity and/or shape of the absorbent assembly during use.

In the illustrated embodiment, the absorbent body 150 of the absorbent assembly 24 is suitably constructed from at least two absorbent body webs (e.g., left web 186a and right web 186b in the cross-section shown in FIG. 3) that each extend longitudinally of the absorbent assembly. Each of the webs 186a, 186b is constructed of the absorbent body material or materials described previously. In particular, one absorbent body web (e.g., the left web 186a) comprises about one-half of the absorbent body 150 and the other absorbent body web (e.g., right web 186b) comprises the other half of the absorbent body 150. The webs 186a, 186b are secured together along a seam 188 that extends at least through the crotch region 126 of the absorbent assembly 24 and may further extend into the front waist region 122 of the absorbent assembly, to the back waist region 124 of the absorbent assembly, or both, depending on the size and intended use of the absorbent body.

The seam 188 suitably extends longitudinally generally at the lateral centerline of the absorbent assembly 24 and at least in part defines the absorbent assembly seam 151. The absorbent body webs 186a, 186b may be secured together by ultrasonic bonding, thermal bonding, adhesive bonding, pressure bonding or other suitable securement technique.

As shown in FIG. 2, the absorbent assembly 24 has a pair of outer edges 193a, 193b at the front waist region 122 thereof and a pair of outer edges 194a, 194b at the back waist region 124 thereof. Opposing outer edges 193a, 194b and 193b, 194b are suitably overlapped and secured together, such as by ultrasonic bonding, thermal bonding, adhesive bonding or other suitable securement technique along a side seam 195 to secure the absorbent assembly in a three-dimensional pants configuration. (In FIG. 2, only the side seam 195 associated with outer edges 193b, 194b is illustrated because the other outer edges 194a, 194b are not secured together.) It is contemplated that instead of being frangibly or permanently secured together, the outer edges 193a, 194a and 193b, 193b may be releasably and refastenably secured together without departing from the scope of this invention.

To further enhance the fit of the absorbent garment 10 on the wearer and to further inhibit leakage of body exudates, the absorbent assembly 24 can also have waist elastic members (not shown) and leg elastic members 196 (FIGS. 2 and 3), as are known to those skilled in the art. The waist elastic members can be operatively joined to the absorbent assembly 24 at the waist ends 138 and 140, such as by attaching the elastic members to the outer cover 146 and/or the bodyside liner 148 while the elastic members are in a stretched condition, so that upon retraction the elastic members gather the absorbent assembly at the waist ends to provide an elastic fit against the wearer's waist. In one embodiment the elastic members can be operatively joined to the absorbent assembly 24, and the elastic members 64 which are operatively joined to the garment shell 22 on laterally opposite sides of the absorbent assembly, together provide an elastic fit of the absorbent garment 10 against substantially the entire waist of the wearer. The elastic members may extend only partially across the respective front and back waist ends 138, 140 of the absorbent assembly 24, or alternatively, the elastic members may extend laterally across the full width of the absorbent assembly 24 at one or both waist ends 138, 140 without departing from the scope of this invention.

The leg elastic members 196 can be operatively joined to the outer cover 146 and/or the bodyside liner 148 and extend longitudinally adjacent the opposite outer edges 193, 194 generally through the crotch region 126 of the absorbent assembly 24.

The waist elastic members (as well as the elastic members 64 operatively joined with the garment shell 22), and the leg elastic members 196 can be formed of any suitable elastic material. As is well known to those skilled in the art, suitable elastic materials include sheets, strands or ribbons of natural rubber, synthetic rubber, or thermoplastic elastomeric polymers. The elastic materials can be stretched and adhered to a substrate, adhered to a gathered substrate, or adhered to a substrate and then elasticized or shrunk, for example with the application of heat, such that elastic retractive forces are imparted to the substrate.

The absorbent assembly 24 can also incorporate other materials or components designed primarily to receive, temporarily store, and/or transport liquid along the mutually facing surface with the absorbent body 150, thereby maximizing the absorbent capacity of the absorbent assembly. For example, one suitable additional component is commonly referred to as a surge layer (not shown). Surge layers are generally well known in the art as being constructed to quickly collect and temporarily hold liquid surges, and to transport the temporarily held liquid to the absorbent body 150.

Various woven and non-woven fabrics can be used to construct the surge layer. For example, the surge layer may be a layer made of a meltblown or spunbond web of synthetic fibers, such as polyolefin fibers. The surge layer may also be a bonded-carded-web or an airlaid web composed of natural and synthetic fibers. The bonded-carded-web may, for example, be a thermally bonded web that is bonded using low melt binder fibers, powder or adhesive. The webs can optionally include a mixture of different fibers. The surge layer may be composed of a substantially hydrophobic material, and the hydrophobic material may optionally be treated with a surfactant or otherwise processed to impart a desired level of wettability and hydrophilicity.

Examples of materials suitable for the surge layer are set forth in U.S. Pat. No. 5,486,166 issued Jan. 23, 1996 in the name of C. Ellis et al. and entitled “FIBROUS NONWOVEN WEB SURGE LAYER FOR PERSONAL CARE ABSORBENT ARTICLES AND THE LIKE”; U.S. Pat. No. 5,490,846 issued Feb. 13, 1996 in the name of Ellis et al. and entitled “IMPROVED SURGE MANAGEMENT FIBROUS NONWOVEN WEB FOR PERSONAL CARE ABSORBENT ARTICLES AND THE LIKE”; and U.S. Pat. No. 5,364,382 issued Nov. 15, 1994 in the name of Latimer et al. and entitled “ABSORBENT STRUCTURE HAVING IMPROVED FLUID SURGE MANAGEMENT AND PRODUCT INCORPORATING SAME”, the disclosures of which are hereby incorporated by reference in a manner consistent with the present document.

The absorbent assembly 24 may also further have a pair of laterally spaced containment flaps (not shown), as are known to those skilled in the art, configured to inhibit the transverse flow of body exudates on the liner to the side edges 193a, 194a and 193b, 194b of the absorbent assembly. For example, the containment flaps may be located generally adjacent to the side edges 193a, 194a and 193b, 194b of the absorbent assembly 24, and can extend longitudinally along the entire length of the absorbent assembly 24 or only partially along the length of the absorbent assembly. Suitable constructions and arrangements for containment flaps are generally well known to those skilled in the art and are described in U.S. Pat. No. 4,704,116 issued Nov. 3, 1987 to Enloe, which is incorporated herein by reference.

In the illustrated embodiment, the absorbent assembly 24 is permanently secured to the garment shell 22 within the interior space thereof. In particular, the front and back waist ends 138, 140 of the absorbent assembly 24 are secured to the garment shell 22 generally at the front and back waist ends 56, 58 thereof, such as by ultrasonic bonding, thermal bonding, adhesive bonding or other suitable securement techniques. It is contemplated, however, that the absorbent assembly 24 may be releasably and refastenably secured to the garment shell 22 to permit detachment and replacement of the absorbent assembly (or detachment and laundering thereof where the absorbent assembly is not disposable) without having to dispose of, launder or otherwise replace the garment shell 22 with a new garment shell.

With the absorbent assembly 24 disposed within and secured to the garment shell 22, the elasticized side edges 136 of the absorbent assembly generally define laterally opposite elasticized leg openings 197 of the absorbent assembly (broadly, inner leg openings of the absorbent garment 10) whereat the absorbent assembly provides an elastic fit against at least part of the wearer's legs. The front and back waist ends 138, 140 of the absorbent assembly 24 together with the waist ends 56, 58 of the garment shell 22 together broadly define the waist opening 20 of the absorbent garment 10. The leg openings 40 of the garment shell 22 broadly define outer leg openings of the absorbent garment 10, separate (e.g., discrete) from the absorbent assembly leg openings 197, whereat the absorbent garment hangs generally loose about the wearer's legs. The attached side margins 48, 50 of the front and back panel assemblies 26, 28 of the garment shell 22 broadly define the side margins 16, 18 of the absorbent garment 10 which are secured along the side seams 19 of the absorbent garment.

FIGS. 4-11 illustrate one embodiment of a process of the present invention for making an absorbent garment such as the absorbent garment 10 of FIGS. 1-3 having an absorbent assembly disposed within and secured to a garment shell. The various drawings illustrate the absorbent garment as being moved by suitable forming apparatus, generally indicated at 200, along a machine direction (indicated by direction arrow X) as it is being configured and assembled. The term “machine direction” thus refers to the direction in which the absorbent garment and/or the various components thereof are moved during forming of the absorbent garment. The term “cross-machine direction” refers to a direction (indicated by direction arrow Y) transverse to the machine direction and generally in the plane of the absorbent garment. An orthogonal or “Z-direction” refers to a direction (indicated by direction arrow Z) that is perpendicular to both the machine direction and cross-machine direction.

With particular reference to FIG. 4, a pair of continuous garment shell webs, indicated generally at 202 and 204, respectively, are fed by respective feeding devices (not shown), such as driven rolls, belt conveyors, chain conveyors, and the like or combinations thereof, to a first securement and cutting station 207 of the forming apparatus 200. In the illustrated embodiment, an upper (with reference to the orientation illustrated in FIG. 4) one of the garment shell webs 202 comprises a pair of continuous garment shell webs 202a, 202b arranged in side-by-side relationship with each other and slightly overlapped as they are fed to the first securement station, generally indicated at 207. A lower one 204 of the garment shell webs comprises a pair of continuous garment shell webs 204a, 204b also arranged in side-by-side relationship with each other and slightly overlapped as they are fed to the first securement station 207.

In the various drawings, the absorbent garment 10 and components thereof are shown as being moved in the machine direction X while in a generally horizontal orientation. Thus, the terms upper and lower are used for reference purposes only in reference to the Z-direction location of absorbent garment components relative to the horizontal orientation of the absorbent garment 10 as it is formed. However, it is understood that the absorbent garment 10 may be moved in the machine direction X while in a generally vertical orientation or other suitable orientation without departing from the scope of this invention.

The continuous lower garment shell webs 204a, 204b are fed to a lower securement device 210 to secure the adjacent webs together along a seam line 212 to thereby define a unitary lower garment shell web 204. The securement device 210 may be an ultrasonic bonding device, thermal bonding device, adhesive bonding device or other suitable securement device, all of which are know to those skilled in the art and need not be set forth in detail herein. The continuous upper garment shell webs 202a, 202b are fed to an upper securement device 215 (suitably similar to the lower securement device) to secure the adjacent webs together along a seam line 217 to thereby define a unitary upper garment shell web 202.

For reference purposes, the garment shell web 202a corresponds to the front left panel member 42a of the garment shell of the absorbent garment shown in FIG. 1; the garment shell web 202b corresponds to the back left panel member 46a; the garment shell web 204a corresponds to the front right panel member 42b; and the garment shell web 204b corresponds to the back right panel member 46b. It is understood that the upper garment shell web 202 may alternatively comprise a one-piece continuous web and/or the lower garment shell web 204 may alternatively comprise a one-piece continuous web without departing from the scope of this invention. It is also contemplated that instead of being continuous, each of the webs 202, 204 may be discrete webs so that discrete absorbent garments are formed by the forming apparatus instead of a continuous web of interconnected absorbent garments that must be subsequently cut into discrete absorbent garments.

The continuous upper and lower garment shell webs 202, 204 are conveyed downstream of the securement devices 210, 215 into opposed, face-to-face relationship with each other as shown in FIG. 4. In particular, outer edges 220 of the upper garment shell web 202 are aligned with outer edges 222 of the lower garment shell web 204. However, it is contemplated that the outer edges 220, 222 of the upper and lower garment shell webs 202, 204 need not be aligned to remain within the scope of this invention. In the illustrated embodiment, the upper and lower garment shell webs 202, 204 are oriented with a respective inner surface 225a, 227a of each web facing outward, i.e., exposed, and respective outer surfaces 225b, 227b of the webs facing each other. The term “inner surface” is used as a reference to the surface of each web 202, 204 that defines the interior space 59 of the garment shell 22 in its finally assembled form (i.e., its closed configuration), the term “outer surface” being used in reference to the surface of each web that forms the exterior surface of the finally formed garment shell.

The opposed garment shell webs 202, 204 are then subjected to a securement operation, and more suitably a bonding operation, in which the garment shell webs are bonded together in a generally asymmetric oval bond pattern 230 extending longitudinally (i.e., in the machine direction X in the illustrated embodiment) of the webs and generally transversely centered thereof as illustrated by bond points in FIG. 4. The oval bond pattern 230 defines a seam line 232 and corresponds to the front and back central seams 44, 47 and the crotch seam (FIG. 1) extending therebetween for each of two consecutive absorbent garments 10 being formed from the webs 202, 204. As shown in FIG. 4, the oval bond pattern 230 is repeated intermittently along the continuous webs 202, 204 for forming a continuous web of absorbent garments 10. It is contemplated that the bond pattern need not be ovate, e.g., it may be D-shaped, rectilinear, diamond-shaped, circular, or other suitable shape without departing from the scope of this invention. It is also contemplated that each oval bond pattern 230 may be a continuous bond instead of the intermittent bond points illustrated in FIG. 4.

A generally oval interior portion 235 of each oval bonded portion of the garment shell webs 202, 204 is cut from the webs, e.g., about and generally adjacent to the oval seam 232 formed by each bond pattern 230, and discarded. In the illustrated embodiment, the forming apparatus 200 comprises a combination bonding and cutting device 240 suitable for concurrently bonding the garment shell webs 202, 204 together in the oval bond pattern 230 and cutting out the interior section 235 from each oval bonded portion.

For example, the bonding and cutting device 240 may comprise an anvil wheel 240a and opposed horn wheel 240b defining a nip 245 through which the garment shell webs 202, 204 are moved in the machine direction X. The wheels 240a, 240b rotate in a facing relationship and use ultrasonic, pressure, or thermal bonding to form the bond pattern 230. Concurrently, a cutting edge 247 formed on at least one of the wheels 240a, 240b cuts the interior portion 235 from the webs 202, 204. The interior portion 235 can be removed by any method known in the art, desirably a vacuum source, (not shown). Such bonding and cutting devices 240 are known to those skilled in the art and will not be further described herein except to the extent necessary to set forth the present invention. With the interior portion 235 removed, a cut-out or crotch opening 249 is thereby formed at the location of the removed interior portion. It is alternatively contemplated that the bonding operation may be performed prior to (e.g., upstream from) the cutting operation, or vice versa, such as by separate bonding and cutting devices (not shown).

The first and second garment shell webs 202, 204 may be provided to and transported through the process by various methods as are well known in the art. For example, the shell webs may be unwound and drawn through the process via driven rolls, belt conveyors, chain conveyors, and the like or combinations thereof.

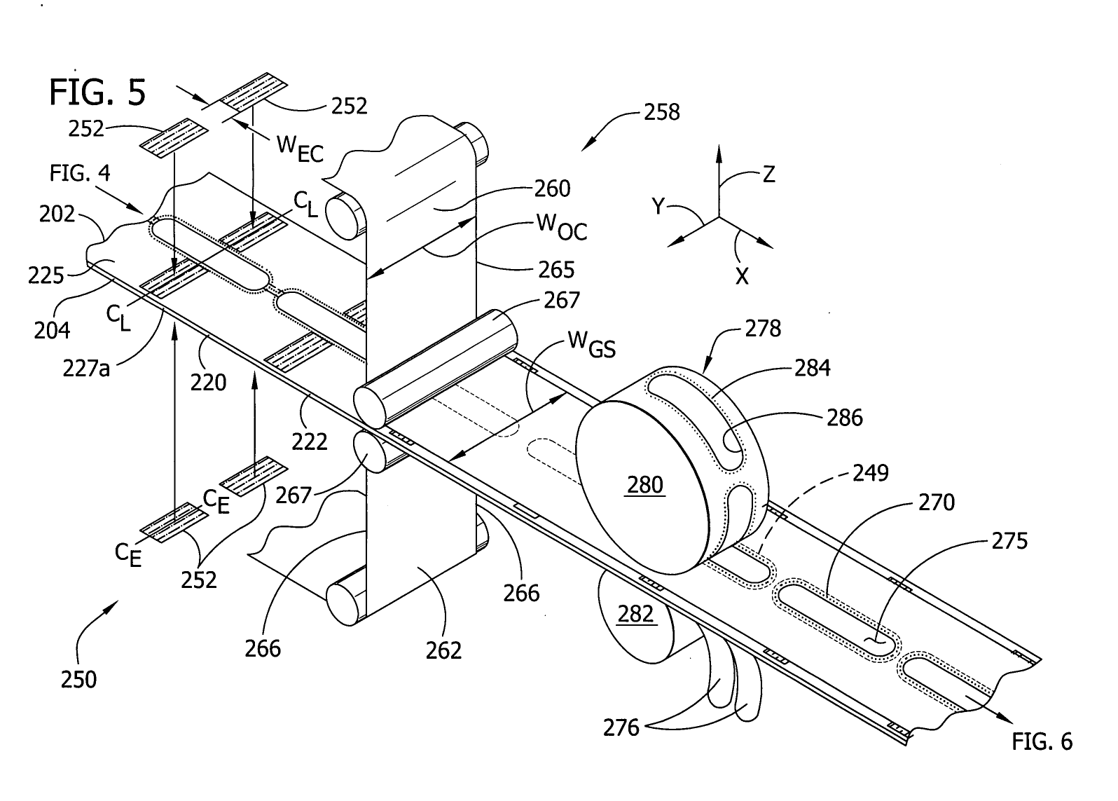

With reference now to FIG. 5, the upper and lower garment shell webs 202, 204 (now secured together) are moved in the machine direction X to a waist elastic station 250 and subjected to an operation in which waist elastic components 252 are applied to the webs 202, 204, e.g., while the webs are still in a generally horizontal (i.e., planar) orientation. The waist elastic components 252 of the illustrated embodiment suitably correspond to the waist elastics 64 (FIG. 2) of two consecutive absorbent garments 10 of the web of absorbent garments 10 being formed. For example, a pair of waist elastic components 252 are positioned on the inner surface 225a, 227a of each of the webs 202, 204 generally at the longitudinal center CL of the oval seam line 232 defined by the bond pattern 230 bonding the two webs together. Each waist elastic component 252 extends transversely (in the cross-machine direction Y of the illustrated embodiment) from the respective outer edge 220, 222 of the respective web 202, 204 generally to adjacent the seam line 232.

Alternatively, a single waist elastic component (not shown) may be applied to the inner surface 225a, 227a of each of the garment shell webs 202, 204 and extend transversely from outer side edge 220, 222 of each web across the crotch opening 249 to the opposite side edge of each web and then later cut from within the crotch opening. It is also contemplated that such a single waist elastic component, or pair of components as in the illustrated embodiment, may be applied to the garment shell webs 202, 204 prior to the bonding operation in which the webs are bonded together about the oval bond pattern 230, or prior to the cutting operation in which the oval crotch opening 249 is cut from the webs, or both. It is understood that waist elastic components 252 may be applied to the inner surface 225a of only the upper garment shell 202, or the inner surface 227a only the lower garment shell 204, without departing from the scope of this invention.

Each waist elastic component 252 is suitably sized in width W_{EC} such that a transverse cut through the garment shell webs 202, 204 and waist elastic components 252, at a widthwise center C_{EC} of the elastic components, results in forming the waist elastics 64 in two consecutive absorbent garments 10 as will be described later herein. The waist elastic components 252 are constructed of any of the suitable materials from which the waist elastics of the absorbent garment 10 may be constructed as described previously. In one embodiment, the waist elastic components 252 may be elongated prior to being applied to the garment shell webs 202, 204. In other embodiments the garment shell webs 202, 204 may be slightly gathered and the waist elastic components 252 may be applied thereto in a generally relaxed condition. The waist elastic components 252 are secured to the respective garment shell webs 202, 204 by ultrasonic bonding, thermal bonding, pressure bonding, adhesive bonding or other suitable securement techniques.

At an outer cover station, generally indicated at 258 (FIG. 5), downstream of the waist elastic station 250, upper (broadly, first) and lower (broadly, second) continuous webs 260, 262 of outer cover material are positioned over and laid upon the inner faces 225a, 227a of the respective upper and lower garment shell webs 202, 204. The outer cover 260, 262 webs of the illustrated embodiment are each suitably sized in width W_{OC} slightly smaller than the width W_{GS} of the garment shell webs 202, 204 but are otherwise sufficiently wide for forming the pants-type absorbent assembly 24 shown in FIGS. 1 and 2. Where the absorbent assembly 24 of the finished absorbent garment 10 is not to be configured for extending fully about the wearer's waist, the outer cover webs 260, 262 may each have a substantially smaller width W_{OC} than as shown in FIG. 5 and remain within the scope of this invention. Preferably, each edge 265, 266 of the outer cover webs 260, 262 is offset from the adjacent edge 220, 222 of the corresponding garment shell web 202, 204.

The outer cover webs 260, 262 may be positioned on the respective garment shell webs 202, 204 in various ways known in the art. For example, in the illustrated embodiment a series of suitable guide rollers 267 are used to position the continuous outer cover webs 265, 267 on the moving garment shell webs 202, 204. The outer cover webs 265, 267 are suitably secured to the waist elastic components 252, such as by adhesive bonding, so as to secure the absorbent assembly 24 to the garment shell 22 before the rest of the absorbent assembly is formed (i.e., the absorbent assembly is formed directly on the garment shell webs 202, 204 instead of the entire absorbent assembly first being formed separate from the garment shell).

For example, the waist elastic components 252 may have an adhesive coating thereon upon application to the garment shell webs 202, 204 or a coating may be applied to the waist elastic components and/or outer cover webs 260, 262 after the waist elastic components are applied to the garment shell webs, such that the outer cover webs adhere to the waist elastic components upon contact of the outer cover webs with the waist elastic components at the rollers 267. It is understood that the outer cover webs 260, 262 may instead, or additionally, be secured to the waist elastic components 252 by ultrasonic bonding, thermal bonding, pressure bonding or other suitable securement techniques. It is also contemplated that the outer cover webs 260, 262 may alternatively, or additionally, be attached to the garment shell webs 202, 204.

The outer cover webs 260, 262 are then subjected to a securement operation and a cutting operation in which the outer cover webs are secured together along a seam line 270 disposed just inward of the seam line 232 along which the garment shell webs 202, 204 are secured (i.e., within the crotch opening 249 of the garment shell webs). The seam line 270 along which the outer cover webs 260, 262 are secured is suitably shaped to correspond to the seam line 232 along which the garment shell webs 202, 204 are secured. It is understood, however, that the shape of the outer cover web seam line 270 need not be shaped similar to the garment shell web seam line 232 to remain within the scope of this invention. An outer cover web crotch opening 275 is formed by cutting away an interior portion 276 of each outer cover web 260, 262 within the seam line 270 (e.g., along and generally adjacent to the seam line) along which the outer cover webs are secured.

The securement operation and cutting operation may be performed concurrently by a suitable bonding and cutting device 278. For example, the bonding and cutting device 278 of the illustrate embodiment comprises an anvil wheel 280 and horn wheel 282 having point bonds 284 and a cutting edge 286 similar to the first bonding and cutting device 240. It is alternatively contemplated that the securement operation may be performed prior to (e.g., upstream from) the cutting operation, or vice versa, such as by separate bonding and cutting devices (not shown). The seam line 270 along which the outer cover webs 260, 262 are secured corresponds to the central seam 158 (FIG. 3) of each absorbent assembly 24 of two consecutive absorbent garments 10 (e.g., waist end-to-waist end) of the web of absorbent garments being formed.

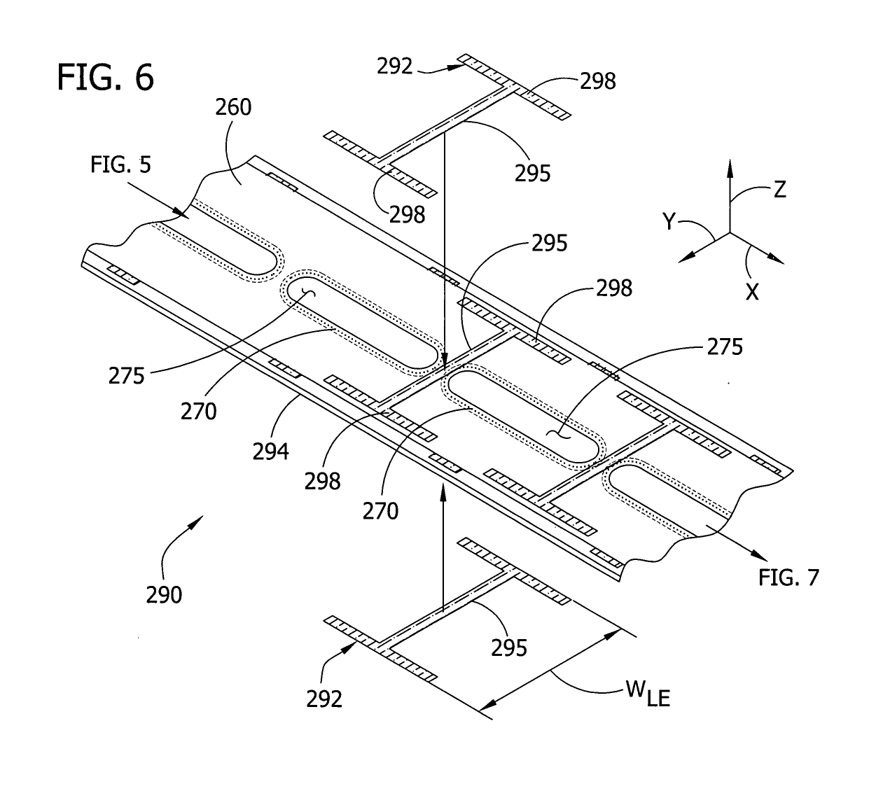

Referring to FIG. 6, at a leg elastic station, generally indicated at 290, downstream of the second bonding and cutting device 278, leg elastic components 292 are applied to inner surfaces 293, 294 of the upper and lower outer cover webs 260, 262. In the illustrated embodiment of FIG. 6, a generally H-shaped leg elastic component 292 is applied to each of the upper and lower outer cover webs 260, 262 with a central segment 295 of each leg elastic component extending transversely (e.g., in the cross-machine direction Y of the illustrated embodiment) across the respective upper and lower outer cover webs between the ends of consecutive seam lines 270 along which the outer cover webs of consecutive absorbent garments 10 (to be formed) are secured. Side segments 298 of each leg elastic component 292 extend longitudinally (e.g., in the machine direction in the illustrated embodiment) on the outer cover webs 260, 262 generally adjacent the outer edges 265 of the outer cover webs. In a particularly suitable embodiment, the side segments 298 of each leg elastic component 292 extend longitudinally upstream and downstream from between the outer cover web seam lines 270 of consecutive absorbent garments 10 to substantially midway along each respective seam line (i.e., adjacent to a position at which the waist elastic component is positioned).

The central segment 295 of each leg elastic component 292 corresponds to a location at which consecutive absorbent garments 10 (crotch-to-crotch) in the web, of absorbent garments is cut. The central segments 295 thus have a width W_{LE} such that a cut taken transversely across the web of absorbent garments 10 through the center of the central segment 295 results in a portion of central segment remaining with each of the two consecutive absorbent garments. It is contemplated that the leg elastic components 292 may be of a shape other than the H-shaped components illustrated in FIG. 6. For example, the leg elastic components 292 may include two elongate strands of elastic material extending in the machine direction and cross-crossing at the locations between consecutive outer cover crotch openings. As another example, the leg elastic components 292 may comprise two adjacent U-shaped components positioned each of the outer cover webs 260, 262 with the closed ends of each pair of components facing each other.

The leg elastic components 292 may be positioned on inner surfaces 293, 294 of the outer cover webs 260, 262 in any suitable manner known in the art. The leg elastics 292 are suitably secured to the outer cover webs 260, 262 such as by being adhesively bonded thereto. However, the leg elastic components 292 may alternatively be secured to the outer cover webs 260, 262 by ultrasonic bonding, thermal bonding, pressure bonding or other suitable securement technique and remain with the scope of this invention.

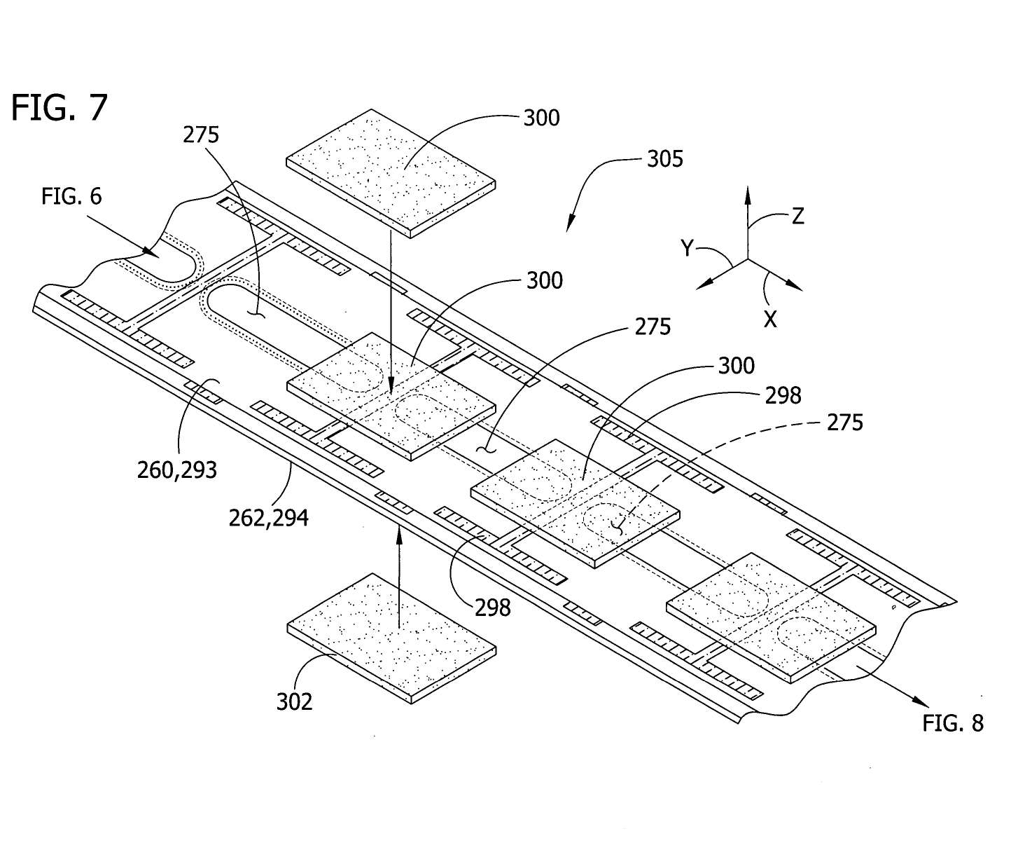

With reference to FIG. 7, discrete absorbent body webs 300, 302 are intermittently laid on and secured to the inner surfaces 293, 294 of each of the upper and lower outer cover webs 260, 262, respectively, at an absorbent body station, generally indicated at 305. Each pair of absorbent body webs 300, 302 (one on the upper outer cover web 260 and the other at generally same location on the lower outer cover web 262) corresponds generally to the absorbent body 150 of the absorbent garment 10 being formed, such as the absorbent garment of FIG. 1-3. For example, the absorbent body webs 300, 302 may be constructed of any of the materials described previously from which the absorbent body 150 of the absorbent garment 10 of FIGS. 1 and 2 may be constructed. In the illustrated embodiment, a discrete absorbent body web 300 is suitably positioned on the upper outer cover web 260 generally centered (both longitudinally and transversely) between consecutive ends of the outer cover crotch opening 275. The absorbent body web 300 extends longitudinally, both upstream and downstream, over a portion of the outer cover crotch opening 275 that extends in the direction of extension of the absorbent body 150.

Another discrete absorbent body web 302 is similarly positioned on the lower outer cover web 262 so that those portions of the upper and lower discrete absorbent body webs that extend over the outer cover crotch openings are in face-to-face, opposed relationship with each other. The upper and lower discrete absorbent body webs 300, 302 are illustrated in FIG. 7 as being generally rectangular. It is understood, however, that the size and/or shape of the absorbent body webs 300, 302 may be other than as illustrated without departing from the scope of this invention. It is also understood that the size, shape and position of each of the discrete absorbent body webs 300, 302 may be determined based on desired characteristics of the finished absorbent garment 10, such as the overall desired absorbency of the garment and the gender of the intended wearer.

The absorbent body webs 300, 302 are suitably secured to the outer cover webs 260, 262 such as by adhesive bonding. It is understood that the absorbent body webs 300, 302 may instead, or additionally, be secured to the outer cover webs 260, 262 by ultrasonic bonding, thermal bonding, pressure bonding or other suitable securement techniques. Additionally, while discrete absorbent body webs 300, 302 are utilized in the illustrated embodiment of FIG. 7, it is contemplated that a pair of continuous absorbent body webs may be used, i.e., secured respectively to the continuous upper and lower outer cover webs 260, 262 such that the absorbent body webs completely cover the outer cover crotch openings.

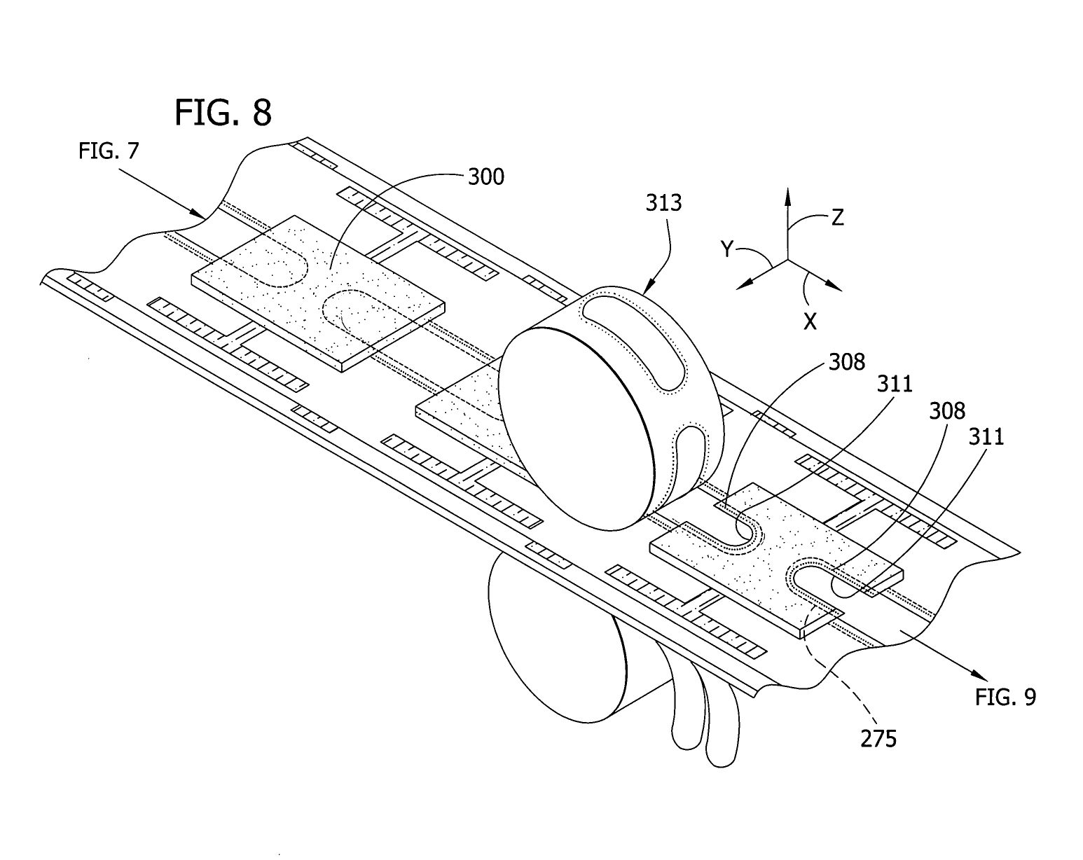

The opposed absorbent body webs are subsequently subjected to a securement operation (FIG. 8) in which the absorbent body webs 300, 302 are secured together within the outer cover crotch openings 275, and to a cutting operation in which a portion of the each absorbent body web within the outer cover web crotch openings is removed from the moving assembly.

In the illustrated embodiment, opposed absorbent body webs 300, 302 (the lower absorbent body web 302 is hidden in FIG. 8) are bonded together along a seam line 308 that generally follows the contour of the outer cover web crotch opening but spaced slightly inward of the outer cover webs at the opening. Each seam line 308 corresponds to the central seam 188 of the absorbent body 150 of one absorbent assembly 24 from one absorbent garment 10 such as is illustrated in FIG. 3 and described previously herein.

In the cutting operation, a portion of each absorbent body web 300, 302 is cut therefrom generally within and adjacent to the seam line 308 along which the absorbent body webs are secured together, forming crotch recesses 311 in the absorbent body webs. While the crotch recesses 311 illustrated in FIG. 8 are generally U-shaped, it is understood that the crotch recesses may be other than U-shaped without departing from the scope of this invention.

In a particularly suitable embodiment, the securement operation and cutting operation for the absorbent body webs 300, 302 are conducted by a third bonding and cutting device, generally indicated at 313, that is substantially similar in construction and operation to the first bonding and cutting device 240 illustrated in FIG. 4 and described previously. It is understood, however, that the securement operation and the cutting operation may be performed by separate, independent devices, and that the cutting operation may be performed prior to the securement operation.

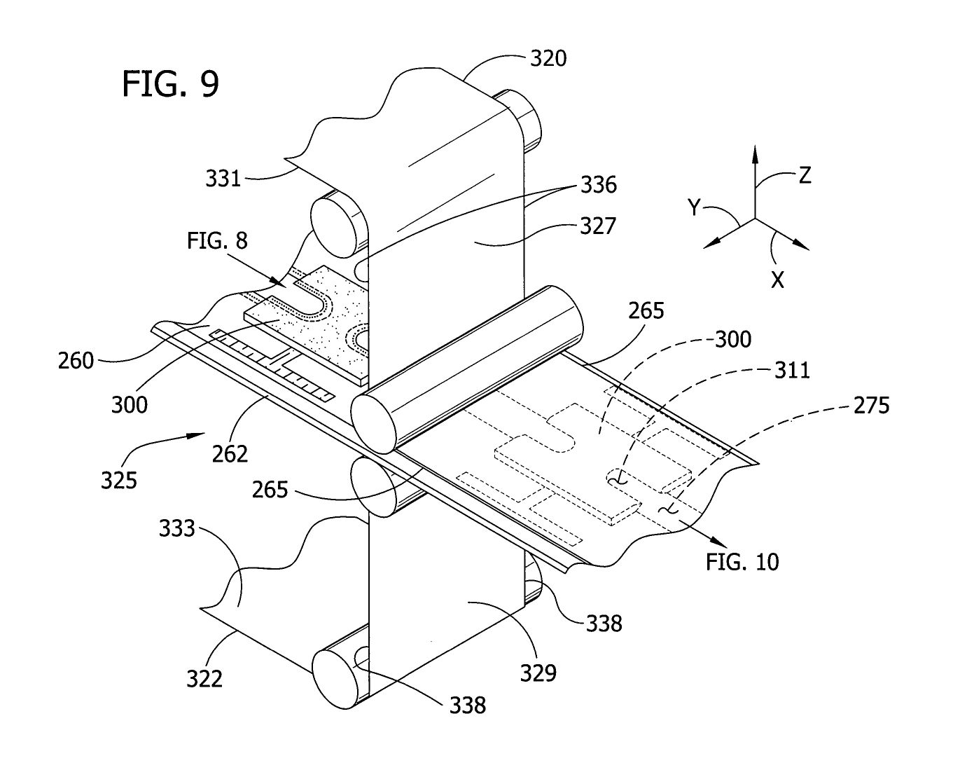

With reference to FIG. 9, upper (broadly, first) and lower (broadly, second) continuous bodyside liner webs 320, 322 are subsequently fed to, positioned over and laid upon the respective absorbent body webs 300, 302 and outer cover webs 260, 262 at a bodyside liner station, generally indicated at 325. The bodyside liner webs 320, 322 together correspond to the bodyside liner 148 of the absorbent assembly 24 of the absorbent garment 10 as illustrated in FIG. 1. Each bodyside liner web 320, 322 has an inner surface 327, 329 (that faces outward away from the absorbent body web 300, 302 and outer cover web 260, 262 but otherwise becomes the inner surface 128 of the absorbent assembly 24 of the finished absorbent garment 10), an outer surface 331, 333 (that faces the absorbent body web and outer cover web), and a pair of opposite side edges 336, 338, respectively.

In the illustrated embodiment, the bodyside liner webs 320, 322 are sized and positioned on the respective outer cover webs 260, 262 such that the side edges 336, 338 of the bodyside liner webs are generally aligned with the respective side edges 265, 266 (side edges 266 are hidden in FIG. 9) of the outer cover webs 260, 262. It is contemplated that the bodyside liner webs 320, 322 may instead be narrower than the outer cover webs 260, 262 as long as the bodyside liner webs are wider than the absorbent body webs 300, 302 so that the absorbent body webs become enclosed by the bodyside liner webs and outer cover webs.

The bodyside liner webs 320, 322 are suitably secured to at least the respective outer cover webs 260, 262 and may additionally be secured to the absorbent body webs 300, 302 as well. Such securement may be by adhesive bonding, ultrasonic bonding, thermal bonding, pressure bonding or other suitable bonding technique. It is also contemplated that the bodyside liner webs 320, 322 may be other than rectangular depending on the desired shape of the absorbent assembly being formed.

As shown in FIG. 9, upon laying the bodyside liner webs 320, 322 over the absorbent body webs 300, 302 and outer cover webs 260, 262 the bodyside liner webs also cover the outer cover crotch openings 275 and extend over the crotch recesses 311 of the absorbent body webs so that the upper and lower bodyside liner webs are in opposed relationship with each other within the crotch recesses of the absorbent body webs.

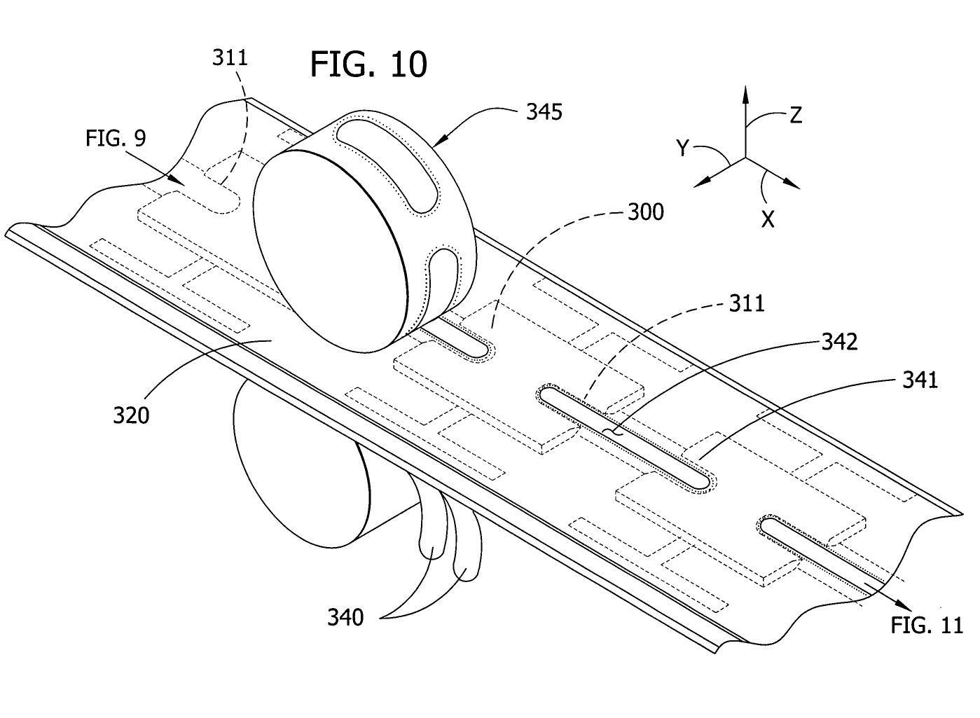

Referring to FIG. 10, in a subsequent securement operation the bodyside liner webs 320, 322 are bonded together along a seam line 341 that generally follows the contour of the absorbent body 150 at the crotch recess 311 but spaced slightly inward of the absorbent body at the recess. Each seam line 341 corresponds to the central seam 176 of the bodyside liner 148 of one absorbent assembly 24 from one absorbent garment 10 such as is illustrated in FIG. 3 and described previously herein. Interior portions 340 of the bodyside liner webs 320, 322 are also cut therefrom within the outer cover crotch openings 275 and crotch recesses 311, generally adjacent the location at which the body side liner webs are secured together, in a cutting operation, thereby forming a third crotch opening 342 through the bodyside liner webs.

In the illustrated embodiment, the securement operation and the cutting operation are performed on the bodyside liner webs 320, 322 by a single bonding and cutting device, generally indicated at 345, similar in construction and operation to the first bonding and cutting device 240 shown in FIG. 4 and described previously. Alternatively, the securement operation and cutting operation may be performed by separate, independent devices. It is also contemplated that the cutting operation may be performed prior to the securement operation without departing from the scope of this invention.

At this stage of the process, both the absorbent garment shell 22 (i.e., the upper and lower garment shell webs 202, 204) and the absorbent assembly 24 that was formed on the garment shell (i.e., the upper and lower outer cover webs 260, 262, absorbent body webs 300, 302, and bodyside liner webs 320, 322) are in what is referred to herein as an open configuration. The term “open configuration” refers to the absorbent assembly 24 and/or the garment shell 22 not being fully assembled in its final three-dimensional configuration (referred to herein as a closed configuration) having a waist opening 20 and at least one leg opening 40. Rather, no waist opening or leg opening has been formed in the open configuration and the inner surfaces 128, 60 of the absorbent assembly 24 and garment shell 22 are generally exposed.

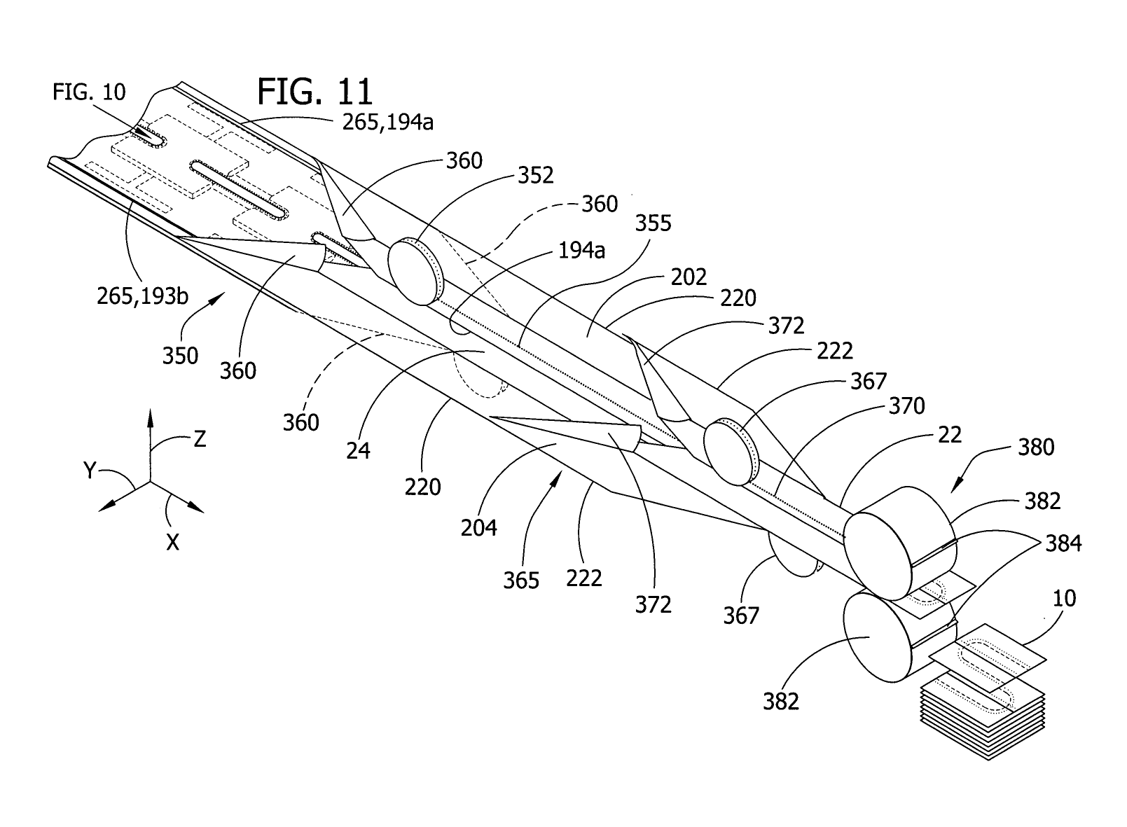

At an absorbent assembly folding station, generally indicated at 350 in FIG. 11, the side edges 193a, 194a and 193b, 194b of the absorbent assembly 24 (i.e., the side edges 265, 266 of the outer cover webs 260, 262 and, in the illustrated embodiment, the side edges 336, 338 of the bodyside liner webs 320, 322 as well) are folded in toward each other and secured together. More particularly, the side edges 193a, 194a of the upper portion of the absorbent assembly 24 (e.g., of the upper outer cover web 260, absorbent body web 300 and bodyside liner web 320) are folded upward (in the illustrated embodiment) and inward (e.g., in the cross-machine direction Y of the illustrated embodiment) so that the side edges generally overlap each other.

The overlapped side edges 193a, 194b are then secured together, such as by a suitable bonding device 352 as shown in FIG. 11, to define a seam 355 that corresponds to the absorbent assembly side seam 195 of the absorbent garment 10 of FIG. 2. The same operation is performed on the lower portion of the absorbent assembly 24 (e.g., of the lower outer cover web 262, absorbent body web 302 and bodyside liner web 322). It is understood that the seam 355 may be formed by adhesive bonding, ultrasonic bonding, thermal bonding, pressure bonding or other suitable bonding technique. Folding of the absorbent assembly side edges 193a, 194a and 193b, 194b may be accomplished by folding skis, which are known to those skilled in the art and are indicated as 360 in FIG. 11, or by other suitable folding techniques. At this stage, the each absorbent assembly 24 of the web of absorbent garments 10 being formed is in its respective closed configuration, with the garment shell 22 still being in an open configuration.

At a subsequent garment shell folding station 365, the side edges 220, 222 of the garment shell webs 202, 204 are folded around the closed absorbent assembly 24 and in toward each other. More particularly, the side edges 220 of the upper garment shell web 202 are folded upward (in the illustrated embodiment) and inward (e.g., in the cross-machine Y direction of the illustrated embodiment) around the closed absorbent assembly 24 so that the side edges of the garment shell web generally overlap each other. The overlapped side edges 220 are then secured together, such as by a suitable bonding device 367, to define a seam 370 that corresponds to the side seams 19 of the absorbent garment 10 of FIG. 2. The same operation is performed on the lower garment shell web 204. It is understood that the seam 370 may be formed by adhesive bonding, ultrasonic bonding, thermal bonding, pressure bonding or other suitable bonding technique. Folding of the garment shell webs 202, 204 may be accomplished by folding skis 372, or by other suitable folding techniques.

It is understood that for embodiments in which the absorbent assembly 24 is not configured to extend about the wearer's waist, the absorbent assembly folding station 350 and operation may be omitted without departing from the scope of this invention.

With the garment shell 22 now in its closed configuration, a web of finished absorbent garments 10 is moved to a separating station, indicated generally at 380 in FIG. 11, where the web of absorbent garments is severed transversely (e.g., in the cross-machine Y direction) across the web to separate the web into discrete absorbent garments. The separating station 380 of the illustrated embodiment suitably comprises opposed cutting rollers 382 having one or more cutting edges 384. However, it is understood that other suitable cutting devices may be used to separate the web into discrete absorbent garments.

When introducing elements of the present invention or the preferred aspect(s) thereof, the articles “a”, “an”, “the”, and “said” are intended to mean that there are one or more of the elements. The terms “comprising,” “including”, and “having” are intended to be inclusive and mean that there may be additional elements other than the listed elements.

As various changes could be made in the above constructions without departing from the scope of the invention, it is intended that all matter contained in the above description or illustrated in the accompanying drawings shall be interpreted as illustrative and not in a limiting sense.

What is claimed is:

1. A process for making an absorbent garment for personal wear about a wearer's waist, the absorbent garment comprising a garment shell having a closed configuration in which the garment shell is adapted to encircle a wearer's waist and has a waist opening, an interior space and at least one leg opening, and an absorbent assembly disposed within the garment shell for taking in and retaining body exudates released by the wearer, the absorbent assembly having an outer cover, a bodyside liner in opposed relationship with the outer cover, and an absorbent body between the liner and the outer cover, said process comprising:
configuring at least one garment shell web in a generally open configuration, said at least one garment shell web having an inner surface and an outer surface;
positioning an outer cover web on the inner surface of said at least one garment shell web while the at least one garment shell web is in said open configuration;
positioning an absorbent body web on said outer cover web;
positioning a bodyside liner web on said absorbent body web, said bodyside liner web having at least one of a length and a width greater than a corresponding one of a length and a width of the absorbent body web such that a portion of the bodyside liner web extends outward of the absorbent body web in generally opposed relationship with the outer cover web;
securing at least a portion of the outer cover web to the inner surface of said at least one garment shell web while said at least one garment shell web is in its open configuration; and
configuring the at least one garment shell web in a closed configuration thereof to define the closed configuration of the garment shell of the absorbent garment, said configuring step comprising folding the garment shell web about the outer cover web, absorbent body web and bodyside liner web such that the inner surface of the at least one garment shell web defines at least in part the interior space of the garment shell and the outer cover web, absorbent body web, and bodyside liner web are disposed within the interior space of the garment shell and together define the absorbent assembly of the absorbent garment.

2. The process set forth in claim 1 wherein the step of securing at least a portion of the outer cover web to the inner surface of the at least one garment shell web is performed prior to the bodyside liner web positioning step.

3. The process set forth in claim 2 wherein the step of securing at least a portion of the outer cover web to the inner surface of the at least one garment shell web is performed prior to the absorbent body web positioning step.

4. The process set forth in claim 1 wherein the absorbent assembly of the absorbent garment has a closed configuration in which the absorbent assembly is adapted to encircle a wearer's waist and defines a waist opening and a pair of leg openings of the absorbent assembly, the process further comprising configuring at least one of the outer cover web and the bodyside liner web in a closed configuration corresponding to the closed configuration of the absorbent assembly of the absorbent garment.

5. The process set forth in claim 1 wherein the step of configuring at least one garment shell web in a generally open configuration comprises configuring a pair of garment shell webs in an open configuration and in opposed relationship with each other, each garment shell web having an outer surface and an inner surface that at least in part subsequently defines the interior space of the garment shell in the closed configuration thereof, said process further comprising securing the opposed garment shell webs together to define at least one seam along which the garment shell webs are secured.

6. The process set forth in claim 5 further comprising cutting away at least a portion of each of the garment shell webs to form a crotch opening in the garment shell webs, the seam line along which the garment shell webs is secured being generally adjacent the crotch opening of the garment shell webs.

7. The process set forth in claim 5 wherein the step of positioning an outer cover web on the inner surface of said at least one garment shell web while the at least one garment shell web is in said open configuration comprises positioning a first outer cover web on the inner surface of one of said garment shell webs and positioning a second outer web on the inner surface of the other one of said garment shell webs, the process further comprising the step of securing the first and second outer cover webs together to define at least one seam along which the outer cover webs are secured.

8. The process set forth in claim 6 wherein the step of positioning an outer cover web on the inner surface of said at least one garment shell web while the at least one garment shell web is in said open configuration comprises positioning a first outer cover web on the inner surface of one of said garment shell webs and positioning a second outer web on the inner surface of the other one of said garment shell webs while the garment shell webs are in an open configuration, the process further comprising the step of securing the first and second outer cover webs together to define at least one seam along which the outer cover webs are secured, said seam being generally within the crotch opening formed in the garment shell webs.

9. The process set forth in claim 7 wherein step of positioning an absorbent body web on the outer cover web comprises positioning a first absorbent body web on the first outer cover web and positioning a second absorbent body web, separate from the first absorbent body web, on said second outer cover web.

10. The process set forth in claim 9 further comprising the step of securing the first absorbent body web to the second absorbent body web to define at least one seam along which the absorbent body webs are secured.

11. The process set forth in claim 10 further comprising, prior to the absorbent body web positioning step, the step of cutting away at least a portion of each of the outer cover webs to form a crotch opening in the outer cover webs, the absorbent body positioning step comprising positioning the first absorbent body web on the first outer cover web such that at least a portion of the first absorbent body web is disposed over at least a portion of the crotch opening in the outer cover webs, and positioning the second absorbent body web on the second outer cover web such that at least a portion of the second absorbent body web is disposed over at least a portion of the crotch opening in the outer cover webs in opposed relationship with said at least a portion of the first absorbent body web, the step of securing the absorbent body webs together comprising securing said at least a portion of the first absorbent body web to said at least a portion of the second absorbent body web.

12. The process set forth in claim 1 further comprising the step of applying a leg elastic component to at least one of said outer cover web and said bodyside liner web, said step being performed after the step of positioning said outer cover web on said at least one garment shell web.

13. The process set forth in claim 1 wherein the at least one garment shell web is continuous for forming a continuous web of a plurality of absorbent garments, said process further comprising separating the continuous web of a plurality of absorbent garments into a plurality of discrete absorbent garments, said separating step being performed after the step of configuring the at least one garment shell web in a closed configuration thereof.

14. A process for making an absorbent garment for personal wear about a wearer's waist, the absorbent garment comprising a garment shell having a closed configuration in which the garment shell is adapted to encircle a wearer's waist and has a waist opening, an interior space and at least one leg opening, and an absorbent assembly disposed within the garment shell for taking in and retaining body exudates released by the wearer, the absorbent assembly having an outer cover, a bodyside liner in opposed relationship with the outer cover, and an absorbent body between the liner and the outer cover, said process comprising:
configuring at least one garment shell web in a generally open configuration, said at least one garment shell web having an inner surface and an outer surface;
positioning a first outer cover web on said at least one garment shell web and positioning a second outer cover web on said at least one garment shell web;
securing at least one of the first and second outer webs to said at least one garment shell web; and
securing the first outer cover web to said second outer cover web to define at least one seam along which the first and second outer cover webs are secured; and
configuring the at least one garment shell web in a closed configuration thereof to define the closed configuration of the garment shell of the absorbent garment, said configuring step comprising folding the garment shell web about the outer cover web such that the inner surface of the garment shell web defines at least in part the interior space of the garment shell and the first and second outer cover webs are disposed within the interior space of the garment shell and at least in part define the absorbent assembly of the absorbent garment.

15. The process set forth in claim 14 further comprising positioning a first absorbent body web on said first outer cover web, and positioning a second absorbent body web, separate from said first outer cover web, on said second outer cover, said first absorbent body web positioning step and said second absorbent body web positioning step being performed prior to said garment shell web configuring step.

16. The process set forth in claim 15 further comprising the step of securing the first absorbent body web to the second absorbent body web to define at least one seam along which the first and second absorbent body webs are secured together.

17. The process set forth in claim 14 further comprising the step of forming an opening in said at least one garment shell prior to the first outer cover web positioning step and the second outer cover web positioning step, said first outer cover web positioning step comprising positioning said first outer cover web on said at least one garment with a portion of the first outer cover web covering at least a portion of the opening in said at least one garment shell web, said second outer cover web positioning step comprising positioning said second outer cover web on said at least one garment with a portion of the second outer cover web covering at least a portion of the opening in said at least one garment shell web and being in opposed relationship with said portion of the first outer cover web.

18. The process set forth in claim 17 wherein the step of securing the first outer cover web to the second outer cover comprises securing the first outer cover to the second outer cover generally within the opening formed in the at least one garment shell web.

19. The process set forth in claim 14 wherein the at least one garment shell web is continuous for forming a continuous web of a plurality of absorbent garments, said process further comprising separating the continuous web of a plurality of absorbent garments into a plurality of discrete absorbent garments, said separating step being performed after the step of configuring the at least one garment shell web in a closed configuration thereof.

20. A process for making an absorbent garment for personal wear about a wearer's waist, the absorbent garment comprising a garment shell having a closed configuration in which the garment shell is adapted to encircle a wearer's waist and has a waist opening, an interior space and at least one leg opening, and an absorbent assembly disposed within the garment shell for taking in and retaining body exudates released by the wearer, the absorbent assembly having an outer cover, a bodyside liner in opposed relationship with the outer cover, and an absorbent body between the liner and the outer cover, said process comprising:
configuring first and second garment shell webs in generally opposed relationship with each other in a generally open configuration of said garment shell webs, said garment shell webs having an inner surface that at least in part defines the interior space of the garment shell in the closed configuration thereof;
positioning a first outer cover web on the inner surface of said first garment shell web;
positioning a second outer cover web, separate from said first outer cover web, on the inner surface of the second garment shell web;
securing at least one of the first and second outer cover webs to at least one of the first and second garment shell webs;
positioning a first absorbent body web on the first outer cover web;
positioning a second absorbent body web on the second outer cover web;
positioning a first bodyside liner web on the first absorbent body web, the first bodyside liner further being in opposed relationship with the first outer cover web;
positioning a second bodyside liner web on the second absorbent body web, the second bodyside liner further being in opposed relationship with the second outer cover web; and
configuring the first and second garment shell webs in a closed configuration thereof to define the closed configuration of the garment shell of the absorbent garment, said configuring step comprising folding the first garment shell web about the first outer cover web, first absorbent body web and first bodyside liner web and folding the second garment shell web about the second outer cover web, second absorbent body web and second bodyside liner web such that the inner surfaces of the garment shell webs at least in part define the interior space of the garment shell and the first and second outer cover webs, first and second absorbent body webs and first and second bodyside liner webs are disposed within the interior space of the garment shell and together define the absorbent assembly of the absorbent garment.

21. An absorbent garment for personal wear about a wearer's waist, said absorbent garment having a longitudinal axis and a lateral axis and comprising:
a garment shell having a closed configuration in which the garment shell is adapted to encircle a wearer's waist and has a waist opening, an interior space and at least one leg opening; and
an absorbent assembly disposed at least in part within interior space of the garment shell for taking in and retaining body exudates released by the wearer, the absorbent assembly having a front waist region, a back waist region and a crotch region extending longitudinally between and interconnecting the front and back waist regions, said absorbent assembly being at least in part secured to the garment shell and comprising an outer cover, a bodyside liner in opposed relationship with the outer cover, and an absorbent disposed between the outer cover and the bodyside liner, said outer cover being constructed of at least two separate webs of material secured together along a seam extending longitudinally generally from the front waist region through the crotch region to the back waist region of the absorbent assembly.

22. The absorbent garment set forth in claim 21 wherein the absorbent body is constructed of at least two separate webs of material secured together along a seam extending longitudinally through the crotch region of the absorbent garment.

23. The absorbent garment set forth in claim 21 wherein the bodyside liner is constructed of at least two separate webs of material secured together along a seam extending longitudinally through from the front waist region through the crotch region to the back waist region.
